(19) United States
(12) Patent Application Publication
(10) Pub. No.: US 2026/0298872 A1
(43) Pub. Date: Oct. 1, 2026

(54) AUTOMATED MULTIPLE LINEAR REGRESSION FOR CALIBRATION CURVES

(71) Applicant: DH Technologies Development Pte. Ltd., Singapore (SG)

(72) Inventors: David Michael COX, Concord (CA); Janna ANICHINA, Concord (CA)

(73) Assignee: DH Technologies Development Pte. Ltd., Singapore (SG)

(21) Appl. No.: 19/478,547

(22) PCT Filed: Apr. 26, 2024
(86) PCT No.: PCT/IB2024/054102

§ 371 Date: Oct. 24, 2025

Related U.S. Application Data
(60) Provisional application No. 63/498,991, filed on Apr. 28, 2023.

(51) Int. Cl. G01N 27/623

(52) U.S. Cl. CPC G01N 27/623

(57) ABSTRACT

A method of calibrating a signal measurement system includes performing a first number of calibration measurements for a first number of samples of a compound, the samples having known concentrations, by measuring a signal for each sample, receiving a signal for a sample having an unknown concentration of the compound; selecting a second number of calibration measurements from the first number of calibration measurements, the second number being smaller than the first number; performing a regression on the selected second number of calibration measurements; and determining the unknown concentration based on the performed regression.

### CROSS-REFERENCE TO RELATED APPLICATION

This application claims the benefit of U.S. Provisional Application No. 63/498,991, filed Apr. 28, 2023, the disclosure of which is hereby incorporated by reference in its entirety.

### BACKGROUND

For large panel assays such as, e.g., quantitative pesticide screens, it is convenient to generate a single set of dilutions to generate a calibration curve that covers a wide range of concentrations. However, some compounds may only show a signal at the high concentration end of the calibration curve, while other compounds may only show a signal in the low concentration end of the calibration curve. In addition, only portions of the calibration curve may be linear. If an unknown sample signal falls within a portion of the calibration curve that is not linear, any estimate of the concentration of the unknown sample may be inaccurate.

### SUMMARY

In one aspect, the technology relates to a method of calibrating a signal measurement system, the method including performing a first number of calibration measurements for a first number of samples of a compound, the samples having known concentrations, by measuring a signal for each sample, receiving a signal for a sample having an unknown concentration of the compound, selecting a second number of calibration measurements from the first number of calibration measurements, the second number being smaller than the first number, performing a regression on the selected second number of calibration measurements, and determining the unknown concentration based on the performed regression.

In another example of the above aspect, selecting the second number of calibration measurements includes dividing the first number of calibration measurements into a plurality of groups of calibration measurements, each group having a smaller number of calibration measurements than the first number, and selecting the group of calibration measurements that encompass the measured signal of the sample having the unknown concentration. For example, each group of calibration parameters includes two to ten measurements of samples having known concentrations. In a further aspect, selecting the second number of calibration measurements includes selecting a first calibration measurement having a higher signal than the received signal and a second calibration measurement having a lower signal than the received signal. For example, the calibration measurements in each group are congruent with one another.

In another example of the above aspect, performing the regression on the selected second number of calibration measurements includes displaying the selecting second number of calibration measurements on a display, generating a curve correlating the second number of calibration measurements on the display, plotting the received signal for the sample having the unknown concentration of the compound on the curve, and extrapolating the concentration of the sample having the unknown concentration from the curve. For example, the first number of known concentrations span over a range of operation of the measurement system. In another example, the signal includes one of a surface area and a signal intensity. In yet another example, the regression is one of a linear regression and a quadratic regression. In other examples, the method further includes storing the first number of calibration measurements in a data repository prior to receiving the signal for the sample having the unknown concentration of the compound. In further examples, performing the first number of calibration measurements includes performing one or more of the first number of calibration measurements after receiving the signal for the sample having the unknown concentration of the compound.

In another aspect, the technology relates to a sample analyzing system that includes a sample ionization device, a sample receiver, a mass analysis device fluidically coupled to the sample receiver and to the sample ionization device, a processor operatively coupled to the sample receiver, the sample ionization device and to the mass analysis device, a data repository operatively coupled to the processor, and a display device operatively coupled to the processor, a memory coupled to the processor, the memory storing instructions that, when executed by the processor, perform a set of operations. In examples, the set of operations includes performing, via the mass analysis device, a first number of calibration measurements for a first number of samples of a compound, the samples having known concentrations, by measuring a signal for each sample, receiving, via the mass analysis device, a signal for a sample having an unknown concentration of the compound, selecting, via the processor, a second number of calibration measurements from the first number of calibration measurements, the second number being smaller than the first number, performing, via the processor, a regression on the selected second number of calibration measurements, and determining, via the processor, the unknown concentration based on the performed regression.

In another example of the above aspect, the instructions include selecting the second number of calibration measurements by dividing the first number of calibration measurements into a plurality of groups of calibration measurements, each group having a smaller number of calibration measurements than the first number, and selecting the group of calibration measurements that encompass the measured signal of the sample having the unknown concentration. For example, each group of calibration parameters includes two to ten measurements of samples having known concentrations. In further example, the instructions include selecting the second number of calibration measurements by selecting a first calibration measurement having a higher signal than the received signal and a second calibration measurement having a lower signal than the received signal. For example, the calibration measurements in each group are congruent with one another.

In further examples, the instructions include performing the regression on the selected second number of calibration measurements by displaying the selecting second number of calibration measurements on the display, generating a curve correlating the second number of calibration measurements on the display, plotting the received signal for the sample having the unknown concentration of the compound on the curve, and extrapolating the concentration of the sample having the unknown concentration from the curve. For example, the first number of known concentrations span over a range of operation of the measurement system. In another example, the signal includes one of a surface area and a signal intensity. In yet another example, the regression is one of a linear regression and a quadratic regression. In further examples, the instructions further include storing the first number of calibration measurements in the data repository prior to receiving the signal for the sample having the unknown concentration of the compound. In another example, the instructions include performing the first number of calibration measurements by performing one or more of the first number of calibration measurements after receiving the signal for the sample having the unknown concentration of the compound. In yet another example, the mass analysis device includes at least one of a differential mobility spectrometer (DMS), a mass spectrometer (MS), and a DMS/MS. In yet a further example, the sample ionization device includes one of a DESI device, a MALDI device, a LAP-MALDI device, a rapid-fire mass spectrometer, a pneumatic ESI device, and an EI device.

### BRIEF DESCRIPTION OF THE DRAWINGS

FIG. 1 is schematic diagram illustrating one example mass analysis system, in accordance with various aspects and examples of the present disclosure.

FIG. 2 is a schematic diagram illustrating an example of a centralized control system, in accordance with various aspects and examples of the present disclosure.

FIG. 3 is a schematic diagram illustrating one particular example of the computing device, in accordance with various aspects and examples of the present disclosure.

FIGS. 4A and 4B depict calibration curves for a plurality of samples of a compound, in accordance with various examples of the disclosure.

FIGS. 5A-5C depict a plurality of measurements using calibration curves, in accordance with various examples of the disclosure.

FIG. 6 depicts a display of a set of recorded calibration data and a corresponding selected calibration curve, in accordance with various examples of the current disclosure.

FIG. 7 represents a flowchart illustrating a method 700 for automated multiple linear regressions, in accordance with various examples of the disclosure.

### DETAILED DESCRIPTION

### Selected Definitions

For the purposes of interpreting this specification, the following definitions will apply and whenever appropriate, terms used in the singular will also include the plural and vice versa. The definitions set forth below shall supersede any conflicting definitions in any documents incorporated herein by reference.

As used herein, the singular forms “a,” “an,” and “the,” include both singular and plural referents unless the context clearly dictates otherwise.

The terms “comprising,” “comprises,” and “comprised of” as used herein are synonymous with “including,” “includes,” or “containing,” “contains,” and are inclusive or open-ended and do not exclude additional, non-recited members, elements, or method steps. It is appreciated that the terms “comprising,” “comprises,” and “comprised of” as used herein comprise the terms “consisting of,” “consists,” and “consists of.”

The recitation of numerical ranges by endpoints includes all numbers and fractions subsumed within the respective ranges, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6, or ≥7, etc. of said members, and up to all said members.

Unless otherwise defined, all terms used in the present disclosure, including technical and scientific terms, have the meaning as commonly understood by one of ordinary skill in the art to which this disclosure belongs. By means of further guidance, term definitions are included to better appreciate the teaching of the present disclosure.

As used herein, “intensity” refers to the height of, or area under, a MS peak. For example, the peak can be output data from a measurement occurring in a mass spectrometer (e.g., as a mass-to-charge ratio (m/z)). In accordance with some examples of the present disclosure, intensity information can be presented as a maximum height of the summary peak or a maximum area under the summary peak representing a m/z value.

Reference throughout this specification to “one example” or “an example” means that a particular feature, structure, or characteristic described in connection with the example is included in at least one example of the present disclosure. Thus, appearances of the phrases “in one example” or “in an example” in various places throughout this specification are not necessarily all referring to the same example, but may. Furthermore, the particular features, structures or characteristics may be combined in any suitable manner, as would be apparent to a person skilled in the art from this disclosure, in one or more examples. Furthermore, while some examples described herein include some, but not other features included in other examples, combinations of features of different examples are meant to be within the scope of the disclosure, and form different examples, as would be understood by those in the art. For example, in the appended claims, any of the claimed examples can be used in any combination.

In the present disclosure, reference is made to the accompanying drawings that form a part hereof, and in which are shown by way of illustration only of specific examples in which the present disclosure may be practiced. It is to be understood that other examples may be utilized, and structural or logical changes may be made without departing from the scope of the present disclosure. The following detailed description, therefore, is not to be taken in a limiting sense, and the scope of the present disclosure is defined by the appended claims.

### Measurement and Analysis Methods and Devices

Sample measurements may be performed on ionized samples. As examples, ionization processes may include Electrospray Ionization (ESI), ionized samples may be generated by Desorption Electrospray Ionization (DESI), which is a combination of ESI and desorption ionization (DI) methods. Another ionization technique may include Matrix-Assisted Laser Desorption Ionization (MALDI), which is an ionization technique that uses a laser energy absorbing matrix to create ions from large molecules with minimal fragmentation. Other ionization techniques may include rapid-fire mass spectrometry, Liquid Atmospheric Pressure MALDI (LAP-MALDI), pneumatic ESI (which generates ions for mass spectrometry using electrospray by applying a high voltage to a liquid to produce an aerosol), and Electron Ionization (EI). Any of the above techniques, as well as others that can perform sample ionization, may be used in examples of this disclosure. The above ionization processes may be combined with an analysis technique such as, e.g., acoustic ejection mass spectrometry (AEMS) or other techniques of sample analysis.

In an additional example, the sample source is a well plate that includes a plurality of wells, and each sample is in one of the plurality of wells. In another example, the mass analysis device includes at least one of a differential mobility spectrometer (DMS), a mass spectrometer (MS), and a DMS/MS.

The systems discussed herein include a central control system that is able to control the underlying subsystems used in the sample analysis process. For example, a script or set of operations may be generated at the central control system or controller that allows for control of the subsystems such that the subsystems are able to work synchronously across different types of operations performed by each of the subsystems. To accomplish such synchronicity across the subsystems, additional mechanical devices, such as robotics, may be incorporated into the overall system to handle transitions of materials between the systems. Thus, the central controller is able to interface with the various subsystems and transition robotics to more efficiently control each of the operations performed by the subsystems. Furthermore, the present systems advantageously include a computing subsystem and various functional modules thereof configured to efficiently process the data generated from multiple samples, reliably determine the data-sample correlation for a large pool of samples, generate mass spectra for each test sample, analyze the generated mass spectra, and provide real-time feedback to other subsystems. As a result, the efficiency and productivity of the entire system may be improved.

Although mass analysis systems coupled with ionization systems are discussed herein, examples of the disclosure can apply to other measurement systems that require calibration of data over a wide range of possible measurements.

### Calibration Technique

In some assays, a large number of samples may be measured via, e.g., a mass analysis system. Due to the large number of samples being analyzed, the calibration or standard curve does not always remain linear across the entire range of concentrations. Current techniques utilize various methods to calibrate a signal, the methods including quadratic regressions, linear regressions, and the like. However, when a signal is non-linear or cover a large range of measurements, even quadratic regressions are not sufficiently accurate to calibrate the signal over the entire range of measurements. The measured signal may be a surface area, a signal intensity, or the like.

For large panel assays (such as, e.g., a quantitative pesticide screen) it is easier to make one set of dilutions for the standard curve that covers a very wide range of concentrations. Some compounds might only show signal at the high concentration standards in this dilution series. Other compounds show signal in the low concentration standards but then saturate the detector in the high concentration standards. Only some regions of the calibration curve could be considered to be linear. When the user, or SCIEX OS automatic calibration curve, selects points to achieve acceptable linearity, some regions of the curve may be excluded. If an unknown sample peak area falls within this region, it may have an inaccurate estimate of its concentration. The sample may need to be re-analyzed using a results table with a calibration curve optimized for its intensity.

For an unknown sample, the peak area would determine which points on the calibration curve are used for linear regression. A better solution may be to find all the points from the calibration curve near this unknown sample intensity that may be used but still achieve acceptable linearity, similarly to what is currently done for automatic calibration curves, but targeting different regions of the calibration curve depending on the intensity of the unknown sample. Each unknown sample may be measured against the region of the calibration curve that is closest to its intensity and that achieves acceptable linearity.

Traditionally, users create a table including multiple results for the same set of samples but for different parts of the calibration curve, and only report the result that matches the concentration of the unknown sample. These can be done with existing software and may require the manual selection of which result that are within the linear range for each analyte.

Examples of the disclosure enable the use of linear regression, which is a generally preferred method for calculating unknown concentration for an unknown sample, without having to custom design the calibration curve for each sample manually.

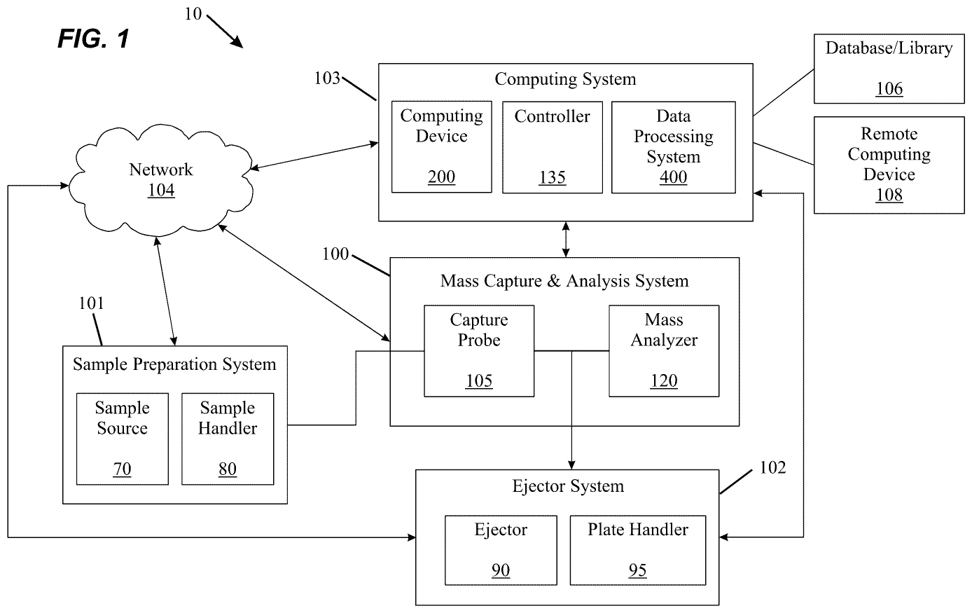

Now referring to FIG. 1, examples of the present disclosure are illustrated and described. In the illustrated examples, the system 10 can each include, in various combinations, pluralities of components, including some or all of: a mass capture and analysis system 100, a sample preparation system 101, an ejector system 102, a computing system 103, a network 104, a database/library 106, and a remote computing device 108. In the example illustrated in FIG. 1, various systems 100, 101, 102, 103, 104, and 106 are subsystems of the system 10 and may be operably connected between or among each other. For example, the computing system 103 is in bilateral communication with the mass capture and analysis system 100, and is also in bilateral communication with the ejector system 102; the sample preparation system 101 is in communication with the mass capture and analysis system 100, and is also in communication with the ejector system 102; the mass capture and analysis system 100 is in communication with the ejector system 102; and the database 106 and the remote computing device 108 are each in communication with the computing system 103.

In some examples, the mass capture and analysis system 100 may be a mass analysis instrument 100. The mass capture and analysis system 100 may be a mass spectrometer system including a mass analyzer 120 for analyzing ions generated from ionization of a sample. The mass capture and analysis system 100 may also include a capture device or probe 105 that captures the sample and provides the sample to other components of the mass capture and analysis system 100. In other examples, the capture probe 105 may be located externally from the mass analysis instrument 100. For instance, the capture probe 105 may be part of the ejection system 102.

It will also be appreciated by a person skilled in the art and in light of the teachings herein that the mass analyzer 120 can have a variety of configurations. Generally, the mass analyzer 120 is configured to process (e.g., filter, sort, dissociate, detect, etc.) sample ions generated by the ion source 115. By way of non-limiting example, the mass analyzer 120 can be a triple quadrupole mass spectrometer, or any other mass analyzer known in the art and modified in accordance with the teachings herein. Other non-limiting, exemplary mass spectrometer systems that can be modified in accordance with various aspects of the systems, devices, and methods disclosed herein can be found, for example, in an article entitled “Product ion scanning using a Q-q-Q linear ion trap (Q TRAP) mass spectrometer” (James W. Hager and J. C. Yves Le Blanc; Rapid Communications in Mass Spectrometry; 2003; 17: 1056-1064); and U.S. Pat. No. 7,923,681, the disclosures of which are hereby incorporated by reference herein in their entireties.

Other configurations, including but not limited to those described herein and others known to those skilled in the art, can also be utilized in conjunction with the systems, devices, and methods disclosed herein. For instance, other suitable mass spectrometers include single quadrupole, triple quadrupole, time-of-flight (ToF), trap, and hybrid analyzers. It will further be appreciated that any number of additional elements can be included in the system 100 including, for example, an ion mobility spectrometer (e.g., a differential mobility spectrometer) that is disposed between the ionization source 115 and the mass analyzer detector 120 and is configured to separate ions based on their mobility difference between in high-field and low-field). Additionally, it is appreciated that the mass analyzer 120 can include a detector 126 that can detect the ions that pass through the analyzer 120 and can, for example, supply a signal indicative of the number of ions per second that are detected.

The sample preparation system 101 may include a sample source 70 and a sample handler 80. The sample source 70 and a sample handler 80 are operative to retrieve collections of samples from the sample source(s) and to deliver the retrieved collections to capture locations associated with sample capture probes 105. The systems may be operative to independently capture selected ones of the pluralities of samples at the capture locations from the pluralities of samples, to optionally dilute the samples and to transfer the captured samples to mass analysis instruments 100, 120 for mass analysis. In some examples, the sample source 70 may include a set of well plates in a storage housing and/or liquid for adding to well plates. The sample source 70 may include part of a liquid handling system that manipulates and/or injects liquid into the well plates. The sample handler 80 includes one or more electro-mechanical devices (e.g., robotics, conveyor belts, stages, etc.) that are capable of transferring the samples (e.g., well plates) from the sample source to other components of the sample preparation system 101 and/or to other systems, such as the ejection system 102 and/or the capture probe 105. As an example, the sample handler 80 may transfer a well plate from the sample preparation system 101 to the ejection system 102. More specifically, the sample handler 80 may transfer the well plate to a plate handler 95 of the ejection system 102. Accordingly, the sample preparation system 101 may also be referred to as a sample delivery system. In some examples, selected sample information (e.g. sample or compound ID, chemical structure of the target compound, or other sample information) could be obtained during the sample handling steps through the use of sample controller 82 and/or the sample handler 80, and communicated to the computing system 103 or the data processing system 400 thereof.

In addition to the plate handler 95, the ejection system 102 may include an ejector 90 that ejects droplets from the wells of the well plates. The ejector 90 may be any type of suitable ejector, such as an acoustic ejector, a pneumatic ejector, or other type of contactless ejector. In an example, the plate handler 95 receives a well plate from the sample handler 80. The plate handler 95 transports the plate to a capture location that may be aligned with the capture probe 105. Once in the capture location, the ejector 90 ejects droplets from one or more wells of the well plates. The plate handler 95 may include one or more electro-mechanical devices, such as a translation stage that translates the well plate in an x-y plane to align wells of the well plate with the ejector 90 and/or or the capture probe 105.

The computing system 103 includes computing resources, components, and modules that are operative to perform various functions including but not limited to: communicating with other subsystems, receiving and transmitting electrical signals with other subsystems or components thereof, receiving, responding to, and executing user instructions, performing calculations, processing raw data received from mass analyzer, performing splitting data, performing sample-dataset correlation, generating and analyzing mass spectrometry data, identifying, annotating, and assigning MS peaks of mass spectra, extracting spectral features from mass spectra, conducting library search, identifying analytes, and outputting analytical report to end users.

In some examples, the computing system 103 includes a computing device 300, a controller 135, and a data processing system 400. The computing device 300 may be in the form of electronic signal processors and operative to perform various computing functions. The controller 135 may be in the form of electronic signal processors and in electrical communication with other subsystems within the system 10. The controller 135 is further configured to coordinate some or all of the operations of the pluralities of the various components of the system 10. The data processing system 400 may include various components and modules operative to process mass spectrometry data and to provide real-time feedback to end users and other subsystems.

In some examples, a network 104 may be operably connected to any one or all of the subsystems or components in the system 10. The network 104 is a communication network. In the example, the network 104 is a wireless local area network (WLAN). The network 104 may be any suitable type of network and/or a combination of networks. The network 104 may be wired or wireless and of any communication protocol. The network 104may include, without limitation, the Internet, a local area network (LAN), a wide area network (WAN), a wireless LAN (WLAN), a mesh network, a virtual private network (VPN), a cellular network, and/or any other network that allows system 104 to operate as described herein.

In some examples, the system 10 may further include one or more library/database 106. The database 106 can be a commercial database, or a private database containing analytical information from previously analyzed samples, or a combination of both. The library/database 106 includes chemical knowledge of standard of known compounds stored therein, including but not limited to chemical formula or elemental composition, neutral mass, monoisotopic mass, or mass of internal fragments thereof. In some examples, the computer system 103 is operative to perform a search using the database 106 and/or to compare data produced by the data processing system 400 to the retrieved data from the database 106 (such as molecular mass information or spectral features) to facilitate mass analysis and/or analyte identification.

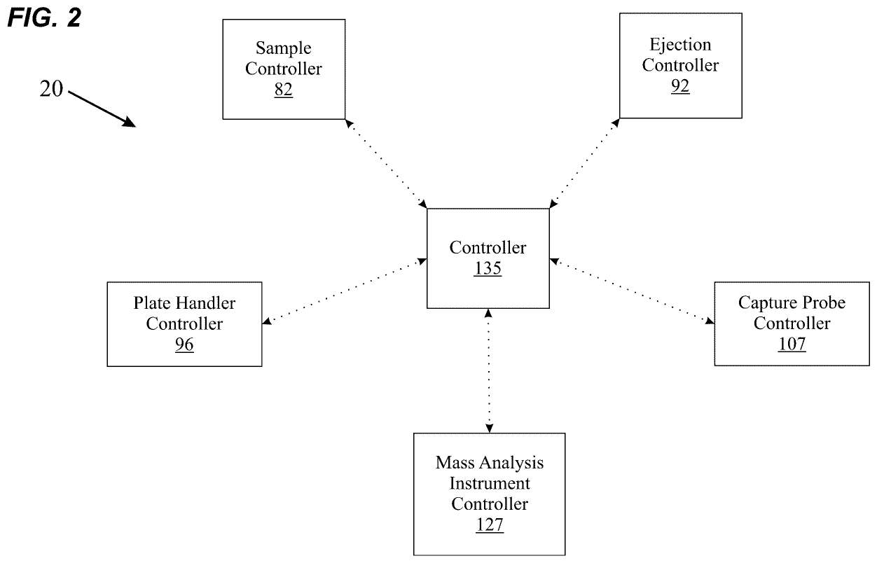

FIG. 2 illustrates one example of a centralized control system 20 for controlling the operation of the system 10, according to FIGS. 1 and 2. In the illustrated example, the centralized control system 20 includes the controllers for each subsystem of the system 10, including 135, 82, 92, 96, 107, and 127. The controller 135 may be a controller for the mass analysis instrument 100 and may be used as the primary controller for controlling components in addition to those components housed within the mass analysis instrument 100. As such, the controller 135 may be considered the main or central controller that orchestrates, or communicates with, the other controllers to carry out the operations discussed herein in a more efficient manner.

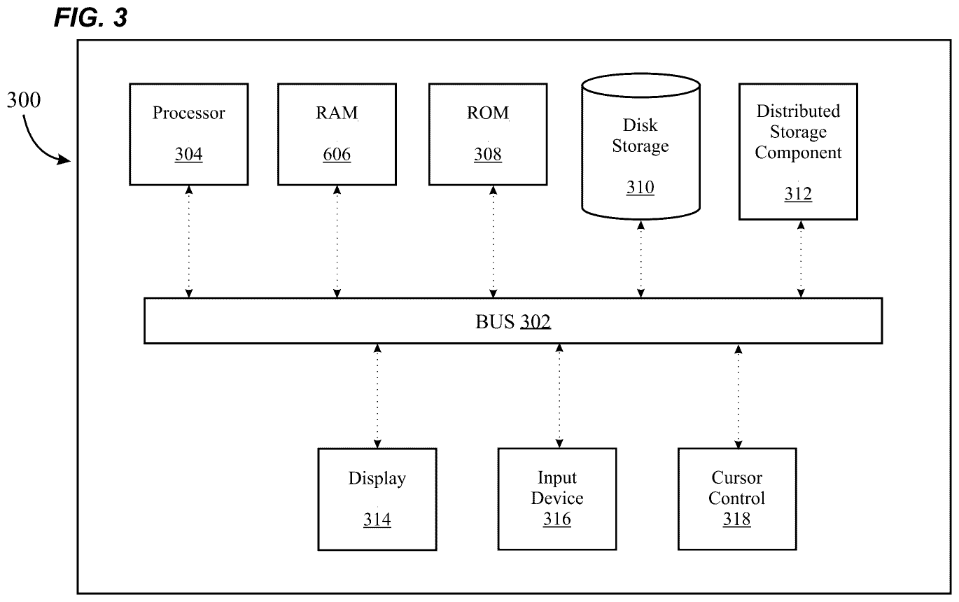

Now referring to FIG. 3, an example of the computing device 300 according to FIGS. 1 and 2 is illustrated and described. It is noted that the computing system 103 of the system 10 may include a single computing device 300 or may include a plurality of distributed computing devices 300 in operative communication with components of a mass analysis instrument 100. In the illustrated example of FIG. 3, the computing device(s) 300 may include a bus 302 or other communication mechanism of similar function for communicating information, and at least one processing element 204 coupled with bus 302 for processing information. As is appreciated by those skilled in the relevant arts, such at least one processing element 204 may include a plurality of processing elements or cores, which may be packaged as a single processor or in a distributed arrangement. Furthermore, in some examples, a plurality of virtual processing elements 204 may be included in the computing device 300 to provide the control or management operations for the mass analysis instrument 100.

Computing device 300 may also include one or more volatile memory(ies) 306, which can for example include random access memory(ies) (RAM) or other dynamic memory component(s), coupled to one or more busses 302 for use by the at least one processing element 204. Computing device 300 may further include static, non-volatile memory(ies) 308, such as read only memory (ROM) or other static memory components, coupled to busses 302 for storing information and instructions for use by the at least one processing element 204. A storage component 310, such as a storage disk or storage memory, may be provided for storing information and instructions for use by the at least one processing element 204. As is appreciated, in some examples the computing device 300 may include a distributed storage component 312, such as a networked disk or other storage resource available to the computing device 300.

Computing device 300 may be coupled to one or more displays 314 for displaying information to a computer user. Optional user input devices 316, such as a keyboard and/or touchscreen, may be coupled to a bus for communicating information and command selections to the at least one processing element 204. An optional graphical input device 318, such as a mouse, a trackball or cursor direction keys for communicating graphical user interface information and command selections to the at least one processing element. The computing device 300 may further include an input/output (I/O) component, such as a serial connection, digital connection, network connection, or other input/output component for allowing intercommunication with other computing components and the various components of the mass analysis instrument 100.

In various examples, computing device 300 can be connected to one or more other computer systems a network to form a networked system. Such networks can for example include one or more private networks, or public networks such as the Internet. In the networked system, one or more computer systems can store and serve the data to other computer systems. The one or more computer systems that store and serve the data can be referred to as servers or the cloud, in a cloud computing scenario. The one or more computer systems can include one or more web servers, for example. The other computer systems that send and receive data to and from the servers or the cloud can be referred to as client or cloud devices, for example. Various operations of the mass analysis instrument 100 may be supported by operation of the distributed computing systems.

Computing device 300 may be operative to control operation of the components of the mass analysis instrument 100 and the sample delivery components 70, 80, 95, 105 through controller(s) 135 and to handle data generated by components of the mass analysis instrument 100 through the data processing system 400. In some examples, analysis results are provided by computing device 300 in response to the at least one processing element 204 executing instructions contained in memory 306 or 308 and performing operations on data received from the mass analysis instrument 100. Execution of instructions contained in memory 306 or 308 by the at least one processing element 204 can render the mass analysis instrument 100 and associated sample delivery components operative to perform methods described herein. Alternatively, hard-wired circuitry may be used in place of or in combination with software instructions to implement the present teachings. Thus, implementations of the present teachings are not limited to any specific combination of hardware circuitry and software.

The term “computer-readable medium” as used herein refers to any media that participates in providing instructions to processor 204 for execution. Such a medium may take many forms, including but not limited to, non-volatile media, volatile media, and transmission media. Non-volatile media includes, for example, optical or magnetic disks, such as disk storage 310. Volatile media includes dynamic memory, such as memory 306. Transmission media includes coaxial cables, copper wire, and fiber optics, including the wires that include bus 302.

Common forms of computer-readable media or computer program products include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 204 for execution. For example, the instructions may initially be carried on the magnetic disk of a remote computer. The remote computer can load the instructions into its dynamic memory and send the instructions over a telephone line using a modem. A modem local to computer system 300 can receive the data on the telephone line and use an infra-red transmitter to convert the data to an infra-red signal. An infra-red detector coupled to bus 302 can receive the data carried in the infra-red signal and place the data on bus 302. Bus 302 carries the data to memory 206, from which processor 304 retrieves and executes the instructions. The instructions received by memory 206 may optionally be stored on storage device 310 either before or after execution by processor 304.

In accordance with various examples, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

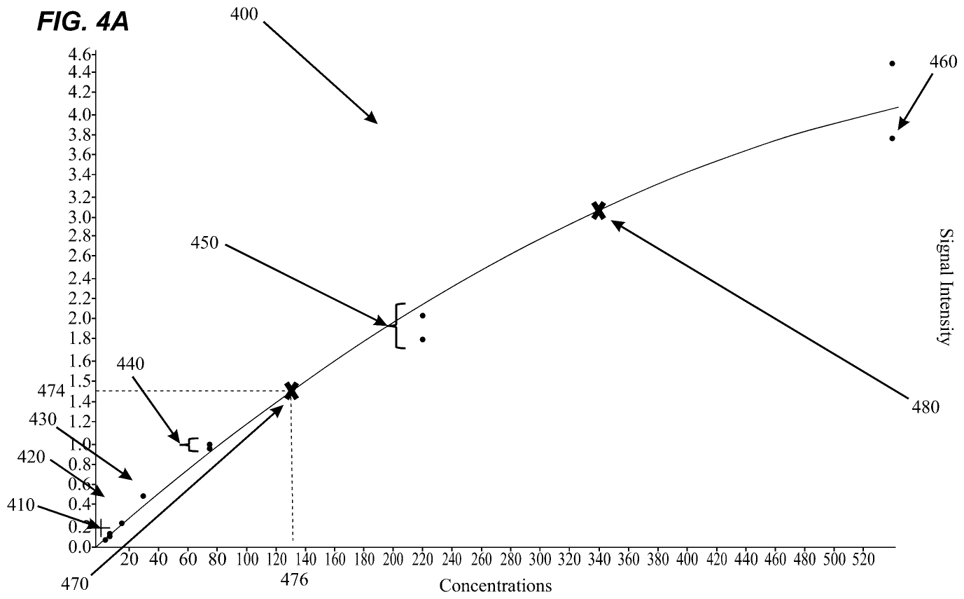

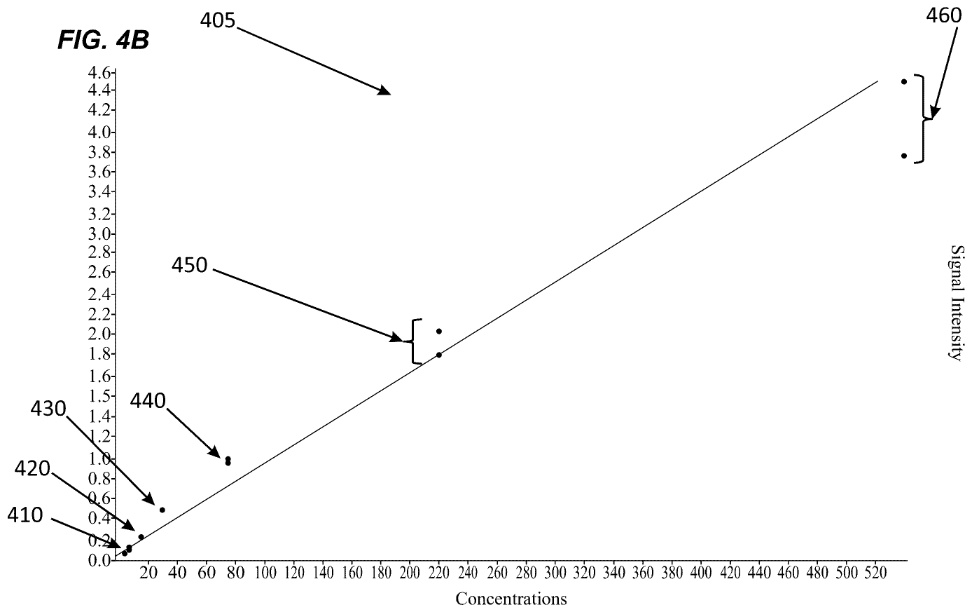

FIGS. 4A and 4B depict calibration curves for a plurality of samples of a compound, in accordance with various examples of the disclosure. In FIG. 4A, a number of measurements 410, 420, 430, 440, 450 and 460 are performed on samples having known concentrations, also referred to herein as “known samples” or standards, and based on the measurements of the known samples, the standard or calibration curve 400 may be generated. In this example, the generated calibration curve 400, also referred to as standard curve 400, is a quadratic curve, and given the wide span of concentrations on the x-axis, the quadratic curve 400 does not sufficiently correlate with all of the measurements 410, 420, 430, 440, 450 and 460. For example, the trajectory of the calibration curve 400 is not sufficiently close to every one of the measurements 410, 420, 430, 440, 450 and 460. In FIG. 4A, the correlation between measurements 410, 420, 430 and 440 and the calibration curve 400 is acceptably close, but the correlation between the calibration curve 400 and measurements for higher concentrations such as, e.g., measurements 450 and 460, is poor. It is typically difficult to accurately correlate all the calibration measurements over the entire range of concentrations, as illustrated in FIG. 4. As a result of the fact that the calibration curve 400 correlates poorly with the measurements of the known samples at the higher concentrations, identifying the concentration of an unknown sample on the x-axis based on the measured signal thereof at those high concentrations may be difficult. In some examples, the measurement for a same concentration may be performed twice or more, as illustrated by the two measurements 440 and 450. Performing more than one measurement for the same concentration may reduce the occurrence of outliers due to artifacts, sampling or pipetting errors, or other extraneous factors.

Identifying the concentration of a sample of unknown concentration is illustrated in FIG. 4A, where two samples of unknown concentrations 470 and 480 are plotted on the calibration curve 400 based on their respective measured intensities. Regarding unknown sample 470, which is bracketed between known samples 440 and 450, since the samples 440 and 450 are well-correlated with the calibration curve 400, and the portion of the calibration curve 400 is almost linear between the measurements 440 and 450, it may be possible to determine the concentration 476 of sample 470 by plotting the experimentally measured intensity 474 of the sample 470 and extrapolating to the corresponding concentration 476. Regarding sample 480, which is bracketed between samples 450 and 460, it appears that the correlation between samples 450 and 460 and the calibration curve 400 is poor. Accordingly, merely plotting the experimentally measured intensity of the unknown sample 480 on the y-axis to extrapolate the corresponding concentration of the unknown sample 480 on the x-axis is unlikely to be accurate.

In FIG. 4B, the calibration or standard curve 405 is a straight line attempting to correlate to measurements 410, 420, 430, 440, 450 and 460. The calibration curve 405 illustrated in FIG. 4B passes by the origin or intersection of the x-axis and the y-axis, and extends linearly. Also in this case, although the correlation between the calibration curve 405 and the first few measurement points 410, 420 and 430 may be acceptable, the correlation between the calibration curve 405 and the remaining measurement points 440, 450 and 460 is poor. Accordingly, calibration curve 405 may only provide accurate concentration measurements, e.g., provides a linear portion, in the range of measurements 410, 420 and 430.

Accordingly, instead of fitting a single quadratic curve or a single linear curve to all of the measurements across the entire range of concentrations, examples of the disclosure include selecting calibration measurements across smaller groups of concentration ranges, for fewer concentration measurements than the entire range of concentrations, and generating linear calibration curves for each group of concentration measurements, the linear calibration curves having a strong correlation to the smaller number of concentrations.

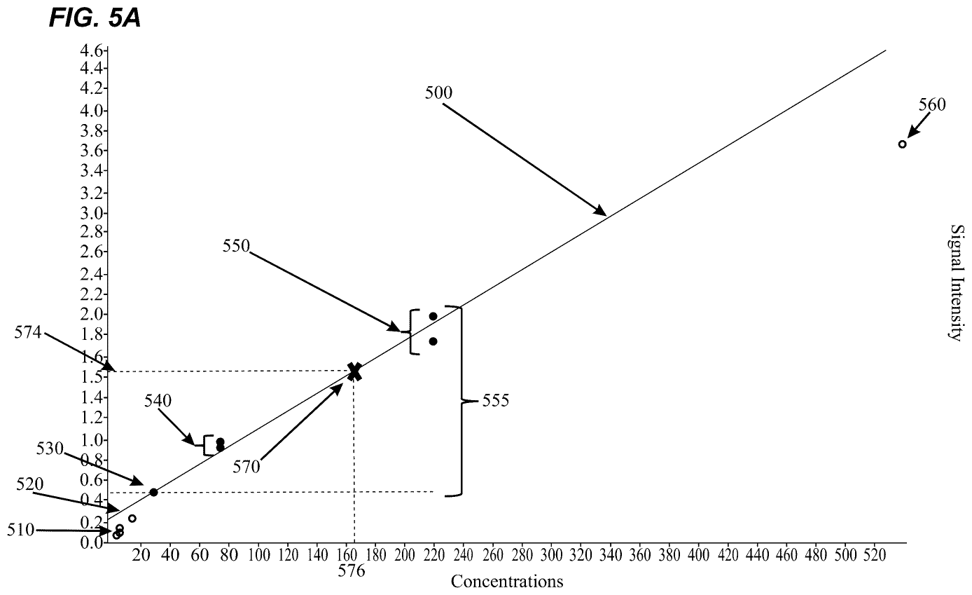

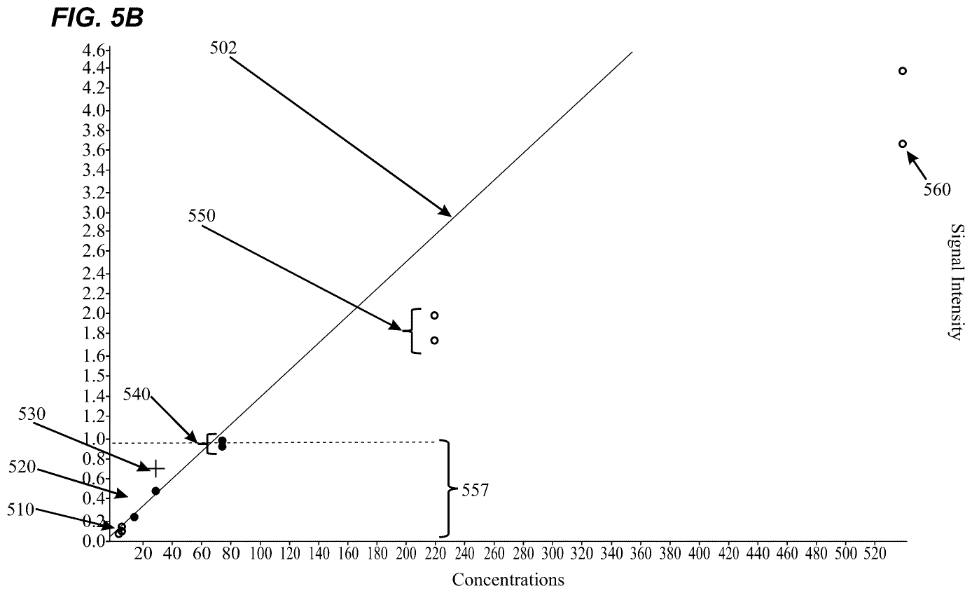

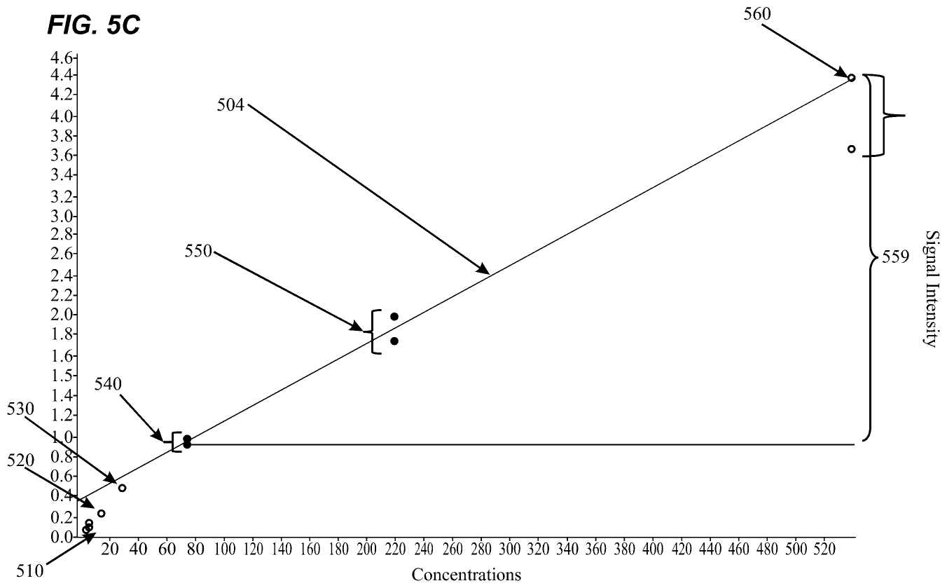

FIGS. 5A-5C depict a plurality of measurements using calibration curves, in accordance with various examples of the disclosure. FIG. 5A depicts a first calibration curve 500 that is generated to correlate three measurement points 530, 540 and 550. In this case, any sample of unknown concentration, herein referred to as unknown sample, having a measured signal in the range 555 illustrated in FIG. 5A, may have the concentration thereof readily identified. Determining the concentration of the unknown sample may be performed by merely plotting the measured signal on the portion of the calibration curve 500 that is within the range 555, e.g., the portion of the calibration curve 500 that is encompassed by the measurements 530, 540 and 550, and extrapolate the corresponding concentration on the x-axis, similarly to the description of FIG. 4A for measurement 470. For example, the measurement 574 for unknown sample 570 may be plotted on the linear portion of the calibration curve 500 that encompasses measurements 530, 540 and 550, the measurement of sample 570 being between within the range 555 that encompasses these measurements. Accordingly, it can be assumed with a degree of confidence that the extrapolated concentration 576 of unknown sample 570 is sufficiently accurate.

FIG. 5B depicts a second calibration curve 502 that is generated to correlate four (4) measurement points 510, 520, 530 and 540. In this case, any unknown sample that has a measured signal in the range 557 illustrated in FIG. 5B, e.g., a measured signal that is lower than the measured signal of the highest measurement point 540, may have the concentration thereof readily determined by merely plotting the measured signal on the portion of the calibration curve 502 that is within the range 557, and extrapolate the corresponding concentration on the x-axis, as discussed above with respect to FIG. 5A.

FIG. 5C depicts a second calibration curve 504 that is generated to correlate three measurement points 540, 550 and 560. Although two measurements have been taken for measurement 560, the calibration curve 504 correlates well with the higher measurement 560. In this case, any unknown sample that has a measured signal in the range 559 illustrated in FIG. 5C, e.g., a measured signal that is encompassed by the measured signals of measurement points 540, 550 and 560, may have the concentration thereof readily determined by merely plotting the measured signal on the portion of the calibration curve 504 that is within the range 559, and extrapolate the corresponding concentration on the x-axis, as discussed above with respect to FIGS. 5A and 5B.

Accordingly, by first generating a plurality of calibration curves, and then selecting one of the generated calibration curves that encompasses the measured signal of an unknown sample, it may be possible to determine the concentration of the unknown sample with good accuracy by plotting the unknown sample on the selected calibration curve and extrapolating the corresponding concentration thereof.

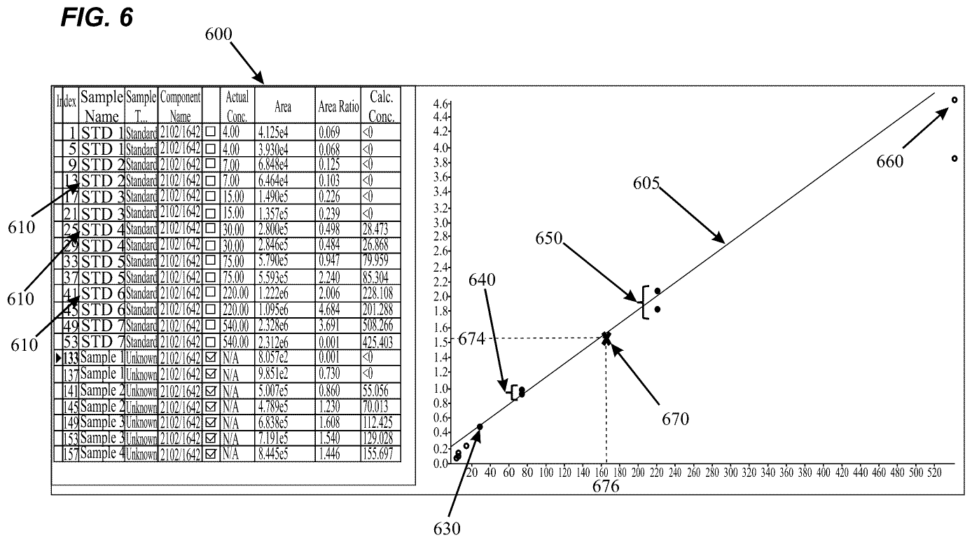

FIG. 6 depicts a display of a set of recorded calibration data and a corresponding selected calibration curve, in accordance with various examples of the current disclosure. In FIG. 6, a table 600 includes a plurality of rows representing calibration parameters 610, each of the calibration parameters 610 being generated by measuring a group of known samples. The group of known samples may include a range of 2 known samples to 10 known samples, and corresponds to a shorter range of concentrations than the entire possible or available range of concentrations that can be measured by the analysis device. Referring back to FIGS. 4A and 4B, the entire range of concentrations spans the entirety of the x-axis and encompasses measurements 410, 420, 430, 440, 450 and 460. In selecting a smaller group of measurements of known samples, it may be possible to obtain an acceptable correlation between a selected calibration curve and an unknown sample. For example, the smaller number of selected samples is illustrated in each of FIGS. 5A, 5B and 5C, which are each a separate selected calibration curve generated based on a number of known samples that is smaller than the total available number of known samples. Each row 610 in table 600 corresponds to a calibration curve generated by performing measurements for a smaller group of known samples.

The known samples selected to generate a calibration curve may be samples that are congruent with each other, e.g., such as samples 530, 540 and 550 that are consecutive to one another in terms of concentration. For example, every one of the measurements on any calibration curve such as curve 500, 502 or 504 is congruent with the other measurements. A calibration curve which measurements are not congruent with each other may be, e.g., a calibration curve correlating measurements 530, 540 and 560 in any one of FIGS. 5A-5C, thus skipping the correlation of measurement 550. These measurement points are not congruent with each other and, in some examples, may not be part of a calibration curve such as the calibration curves listed in table 600.

In operation, as illustrated in FIG. 6, a number of calibration measurements 610 are generated, the calibration measurements being measurements of small groups of known samples, e.g., smaller than the entire range of possible known sample concentrations. Once the calibration measurements 610 are performed, stored, and displayed in table 600, then the measurement signal for an unknown sample 670 is performed, with a measured signal 674. When the signal 674 is measured and possibly recorded, one of the calibration parameters 610 listed in the table 600 is selected so as to encompass the measured signal 674. For example, the selected calibration parameters 610, illustrated in blue in FIG. 6, includes measurement signals of known samples which are close to, or closest to, the measured signal 674. In the example illustrated in FIG. 6, the closest measurements are those for measurements 630, 640 and 650. Accordingly, the displayed calibration curve 605, which corresponds to the selected calibration parameters 610 and measurements 630, 640 and 650, is displayed on a display screen, and the measurement 674 of the unknown sample 670 is plotted on the displayed calibration curve 605. Based on the plotted measurement 674 of the unknown sample 670 on the displayed calibration curve 605, the concentration value 676 of the unknown sample 676 may be extrapolated and determined.

The choice of which calibration curve to use is relevant to obtaining a sufficiently accurate correlation, and thus a sufficiently accurate measurement of the concentration of the unknown sample. Choosing the calibration curve such as calibration curve 605 illustrated in FIG. 6 may be performed by choosing known samples which measurement signals are closest to the measurement signal of the unknown sample. Accordingly, if the calibration measurement 605 has three measurements, two of the measurements may have concentrations that are above the measured concentration of the unknown sample, and one of the measurements, may have a concentration that is below the measured concentration of the unknown sample. Conversely, two of the measurements may have concentrations that are below the measured concentration of the unknown sample, and one of the measurements may have a concentration that is above the measured concentration of the unknown sample. In other examples, all three measurements of the calibration curve may be above, or below, the measured signal of the unknown sample. In addition, although the example illustrated in FIG. 6 is an example of a calibration curve having three measurements, calibration curves may be generated by other numbers of measurements such as, e.g., two, three, four, five or more measurements, as longs as the measurements of the know samples are congruent with each other, and the correlation between the calibration curve and each of the known sample is sufficiently acceptable.

In various examples, the selection of the calibration parameters 610 may be performed manually by, e.g., a user, but may also be performed automatically. During the automatic selection of the calibration parameters 610, each calibration parameter 610 may be examined to determine which calibration parameter 610 includes intensity measurement that are closest to the measurement of the unknown sample while remaining congruent with one another. Accordingly, a processor may automatically determine which calibration parameter 610 is best suited to determine the concentration of an unknown sample based on the measured intensity of the unknown sample.

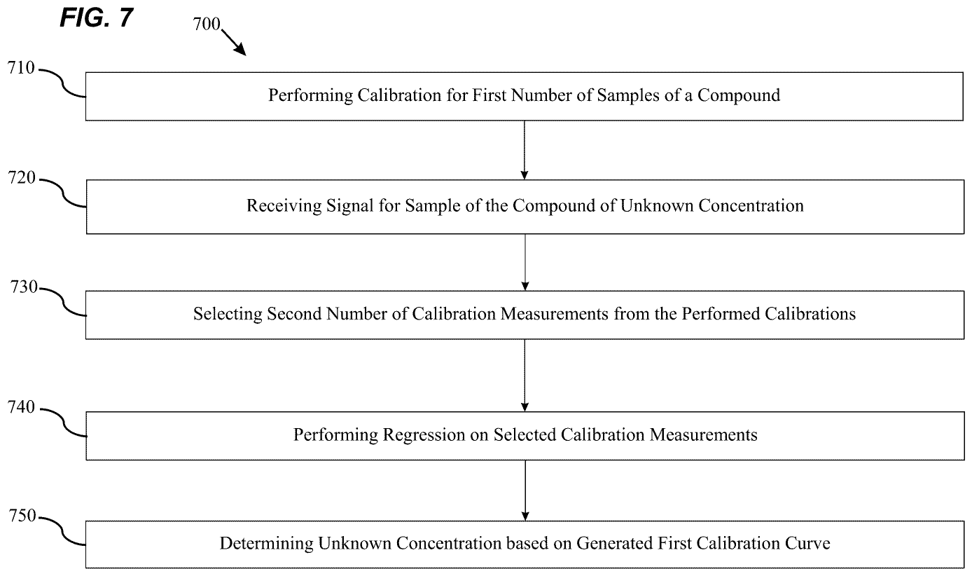

FIG. 7 represents a flowchart illustrating a method 700 for automated multiple linear regressions, in accordance with various examples of the disclosure. The method 700 includes operation 710, during which a plurality of calibration measurements are performed for a total or first number of samples of a compound to generate a calibration curve. The samples have known concentrations, and the calibration is performed by measuring one or more signals for each known concentration. Measuring more than one signal for each concentration allows to eliminate or reduce the occurrence of false results or outliers, results altered by an artifact, sampling or pipetting errors, or other accidental results. The first number of known concentrations may span over a range of operation of the measurement system, from a lowest detectable signal or concentration to a highest detectable signal or concentration, and form a calibration curve that spans the entire range of samples. The calibration curve is typically non-linear in shape, but includes smaller linear portions therein. In an example, operation 710 includes storing the first number of calibration measurements in a data repository prior to performing operation 720 discussed below.

Operation 720 includes measuring the signal for a sample having an unknown concentration, or unknown sample. The sample which signal is measured during operation 720 is the same compound as the samples used to generate the calibration curves during operation 710, but has an unknown concentration. In an example, one or more of the first number of calibration measurements during operation 710 are performed after receiving the signal for the sample having the unknown concentration of the compound during operation 720.

Operation 730 includes selecting a second number of calibration measurements from the calibration measurements performed during operation 710. The first number of calibration measurements may be divided into a plurality of groups of measurements, each group having a smaller number of calibration measurements than the total or first number of measurements. For one of the groups, the second number of calibration measurement that form the group may be, e.g., in a range of two to ten. During operation 730, selecting the second number of calibration measurements includes selecting one of the groups of calibration measurements. In each group of calibration measurements, the calibration measurements are congruent with each other and are linearly oriented with respect to each other in a portion of the calibration curve generated during operation 710.

The selection of the second number of calibration measurements during operation 730 is based on the signal of the unknown sample that was measured during operation 720. For example, selecting the second number of calibration measurements includes dividing the first number of calibration measurements into a plurality of groups of calibration measurements, each group having a smaller number of calibration measurements than the first number, and selecting the group of calibration measurements that encompass the measured signal of the sample having the unknown concentration. The selected group may include a signal that is higher than the signal of the unknown sample received during operation 720, and a signal that is lower than the signal of the unknown sample received during operation 720. Selecting the second number of calibration measurements may include selecting calibration measurements that have signals that are within proximity of the measured signal of the unknown sample. For example, the signals of the selected calibration measurements are within a range of 10-30% of the measured signal of the unknown sample. Selecting the second number of calibration measurements may also include selecting calibration measurements that are consecutive to, or congruent with, one another on the calibration curve generated during operation 710. Selecting the second number of calibration measurements may further include selecting calibration measurements having signals that are closest to the signal of the unknown sample measured during operation 720.

Operation 740 includes performing a regression on the selected number of calibration measurements. For example, the selected number of calibration measurements may be displayed as a calibration curve, as displayed in FIG. 6, and discussed above. Based on the displayed selected calibration measurements which are linearly oriented with respect to one another, the regression may be performed. With respect to FIG. 6, the calibration parameters 610 that correspond to the measurements 630, 640 and 650 that are closest to the measurement 670 of the unknown sample is selected, and the measurements 630, 640 and 650 are displayed on a display screen. Performing the regression during operation 740 may include displaying the selecting second number of calibration measurements on a display, generating a curve correlating the second number of calibration measurements on the display, plotting the received signal for the sample having the unknown concentration of the compound on the curve, and extrapolating the concentration of the sample having the unknown concentration from the curve. The regression may be a linear regression or a quadratic regression.

Operation 750 includes determining the unknown concentration based on the generated first calibration curve. With reference to FIG. 6, the concentration of the unknown sample 670 is determined by plotting the measured signal 674 of the unknown sample on the displayed calibration curve 605, and extrapolating the concentration 676.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although specific examples were described herein, the scope of the technology is not limited to those specific examples. One skilled in the art will recognize other examples or improvements that are within the scope of the present technology. Therefore, the specific structure, acts, or media are disclosed only as illustrative examples. Examples according to the technology may also combine elements or components of those that are disclosed in general but not expressly exemplified in combination, unless otherwise stated herein. The scope of the technology is defined by the following claims and any equivalents therein.

1. A method of calibrating a signal measurement system, the method comprising:
performing a first number of calibration measurements for a first number of samples of a compound, the samples having known concentrations, by measuring a signal for each sample;
receiving a signal for a sample having an unknown concentration of the compound;
selecting a second number of calibration measurements from the first number of calibration measurements, the second number being smaller than the first number;
performing a regression on the selected second number of calibration measurements; and
determining the unknown concentration based on the performed regression.

2. The method of claim 1, wherein selecting the second number of calibration measurements comprises:
dividing the first number of calibration measurements into a plurality of groups of calibration measurements, each group having a smaller number of calibration measurements than the first number; and
selecting the group of calibration measurements that encompass the measured signal of the sample having the unknown concentration.

3. (canceled)

4. The method of claim 1, wherein selecting the second number of calibration measurements comprises selecting a first calibration measurement having a higher signal than the received signal and a second calibration measurement having a lower signal than the received signal.

5. The method of claim 2, wherein the calibration measurements in each group are congruent with one another.

6. The method of claim 1, wherein performing the regression on the selected second number of calibration measurements comprises:
displaying the selecting second number of calibration measurements on a display;
generating a curve correlating the second number of calibration measurements on the display;
plotting the received signal for the sample having the unknown concentration of the compound on the curve; and
extrapolating the concentration of the sample having the unknown concentration from the curve.

7. The method of claim 1, wherein the first number of known concentrations span over a range of operation of the measurement system.

8. The method of claim 1, wherein the signal comprises one of a surface area and a signal intensity and wherein the regression is one of a linear regression and a quadratic regression.

9. (canceled)

10. The method of claim 1, further comprising storing the first number of calibration measurements in a data repository prior to receiving the signal for the sample having the unknown concentration of the compound.

11. The method of claim 1, wherein performing the first number of calibration measurements comprises performing one or more of the first number of calibration measurements after receiving the signal for the sample having the unknown concentration of the compound.

12. A sample analyzing system comprising:
a sample ionization device;
a sample receiver;
a mass analysis device fluidically coupled to the sample receiver and to the sample ionization device;
a processor operatively coupled to the sample receiver, the sample ionization device and to the mass analysis device;
a data repository operatively coupled to the processor; and
a display device operatively coupled to the processor;
a memory coupled to the processor, the memory storing instructions that, when executed by the processor, perform a set of operations comprising:
performing, via the mass analysis device, a first number of calibration measurements for a first number of samples of a compound, the samples having known concentrations, by measuring a signal for each sample;
receiving, via the mass analysis device, a signal for a sample having an unknown concentration of the compound;
selecting, via the processor, a second number of calibration measurements from the first number of calibration measurements, the second number being smaller than the first number;
performing, via the processor, a regression on the selected second number of calibration measurements; and
determining, via the processor, the unknown concentration based on the performed regression.

13. The system of claim 12, wherein the instructions comprise selecting the second number of calibration measurements by:
dividing the first number of calibration measurements into a plurality of groups of calibration measurements, each group having a smaller number of calibration measurements than the first number; and
selecting the group of calibration measurements that encompass the measured signal of the sample having the unknown concentration.

14. (canceled)

15. The system of claim 12, wherein the instructions comprise selecting the second number of calibration measurements by selecting a first calibration measurement having a higher signal than the received signal and a second calibration measurement having a lower signal than the received signal.

16. The system of claim 13, wherein the calibration measurements in each group are congruent with one another.

17. The system of claim 12, wherein the instructions comprise performing the regression on the selected second number of calibration measurements by:
displaying the selecting second number of calibration measurements on the display;
generating a curve correlating the second number of calibration measurements on the display;
plotting the received signal for the sample having the unknown concentration of the compound on the curve; and
extrapolating the concentration of the sample having the unknown concentration from the curve.

18. The system of claim 12, wherein the first number of known concentrations span over a range of operation of the measurement system.

19. The system of claim 12, wherein the signal comprises one of a surface area and a signal intensity, and wherein the regression is one of a linear regression and a quadratic regression.

20. (canceled)

21. The system of claim 12, wherein the instructions further comprise storing the first number of calibration measurements in the data repository prior to receiving the signal for the sample having the unknown concentration of the compound.

22. The system of claim 12, wherein the instructions comprise performing the first number of calibration measurements by performing one or more of the first number of calibration measurements after receiving the signal for the sample having the unknown concentration of the compound.

23. The system of claim 12, wherein the mass analysis device comprises at least one of a differential mobility spectrometer (DMS), a mass spectrometer (MS), and a DMS/MS.

24. (canceled)
